(19) United States
(12) Patent Application Publication
(10) Pub. No.: US 2005/0123496 A1
(43) Pub. Date: Jun. 9, 2005

(54) Self foaming non-aqueous personal wash cleansers with little or no surfactant

(75) Inventors: Pravin Shah, Rutherford, NJ (US); Vivek Subramanian, Livingston, NJ (US); Judith Lynne Kerschner, Hawthorne, NJ (US); Xiaodong Zhang, Livingston, NJ (US); Rajendra M. Dave, Newark, NJ (US); Frank Meyer, Fort Lee, NJ (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/730,545

(22) Filed: Dec. 8, 2003

(51) Int. Cl. A61K 7/075; A61K 7/08

(52) U.S. Cl. 424/70.22

(57) ABSTRACT

The invention relates to non-aqueous personal wash compositions comprising little or no surfactant but which utilize a foam enhancing system to generate good levels of foam.

### FIELD OF THE INVENTION

The present invention provides personal wash cleansing compositions with little or no surfactant, yet still able to deliver foam. The compositions are non-aqueous compositions containing two or more reactants which are non-reactive with each other in the non-aqueous liquids, but which react to generate foam when the non-aqueous composition is diluted during wash.

### BACKGROUND

It is known to use reactants to generate foam upon mixing. However, the reactive components are either used in aqueous systems and separated by dual chamber container until use (so they will not react in the aqueous system before use); or the solid reactants are enclosed in a cleansing “pillow” and applied in a form of wipe rather than as a traditional liquid shower cleanser.

U.S. Pat. No. 5,804,546 to Hall, for example, discloses aqueous cleansing formulations comprising surfactant and two reacting components which, when mixed together, generate a gas which provides lather. The two components must be kept in two separate chambers (versus single chamber products of the subject invention) to avoid their reacting in situ.

U.S. Pat. No. 6,063,390 to Farrell et al. discloses a cleansing pillow containing solid sodium bicarbonate, solid acid and a solid surfactant enclosed in a “pillow”. When the pillow is wet with water, the solids dissolve and form a lather for cleansing.

Applicants are aware of no art which discloses non-aqueous liquids for dispensing solids (solids being non-reactive in the non-aqueous liquid found in a single-chamber product) such that the liquid product can be used as a “traditional” shower liquid.

### BRIEF DESCRIPTION OF THE INVENTION

The present invention comprises a non-aqueous liquid personal wash cleanser product in a single-chamber receptacle which cleanser comprises two or more reactants which will generate foam when the non-aqueous cleanser is diluted with water. The cleanser preferably contains little or no surfactant but, because of the reactants, provides good foam (e.g., greater than 25 cc after 30 seconds using protocol described herein).

More specifically, the invention comprises non-aqueous personal wash cleanser comprising:
(1) 0 to 25%, preferably 0.5 to 25%, more preferably 1.0 to 15%, more preferably 2 to 10% and more preferably no more than about 10% by wt. of a surfactant selected from the group consisting of anionic, nonionic, amphoteric/zwitterionic, cationic surfactant and mixtures thereof;
(2) 15 to 50% by wt. of a foam enhancing system comprising two or more reactants non-reactive in a non-aqueous medium in which they are carried but which react, when diluted in water, to generate at least about 25 cc, more preferably at least about 30 cc of foam after 30 seconds following a defined foam measurement profile;
(3) 30 to 70% of said non-aqueous medium defined by the fact that the reactants of (2) are non-reactive (i.e., won't react to form foam in the medium) with each other in said medium. In some embodiments, one or both reactants are preferably, although not necessarily insoluble or sparingly soluble in the medium;
(4) 0 to 10%, preferably 1 to 10% polyalkylene glycol; and
(5) 0 to 10%, preferably 0.5 to 5% liquid thickener (preferably hydrophobic and thickener although it can be a hydrophilic thickener since the medium may have hydrophobic and hydrophilic characteristics).

Using the foam enhancing system in the non-aqueous system defined above, it is possible to provide good levels of foam from a single-chamber unit while simultaneously utilizing low levels of surfactant.

In a second embodiment of the invention, foam production is even further enhanced when the non-aqueous medium further comprises polyalkylene glycol, preferably 1 to 10% alkyleneglycol.

### DETAILED DESCRIPTION OF THE INVENTION

The present invention relates to novel compositions and processes for delivering good foam from a low or no surfactant cleansing liquid.

Normally, good foam can be delivered from surfactant present in a surfactant containing cleanser. The surfactant, however, also can be irritating to the skin. It is thus desirable to find a way to deliver foam in a cleanser even when little or no surfactant is present.

This can be accomplished, for example, using reactants which will combine to generate gas/foam. For example, sodium bicarbonate and citric or lauric acid, when combined will release gas/foam.

Because, as noted, these gas forming reactants will readily foam when placed in a typical aqueous cleanser, the art has disclosed the use of such reactants whereby the reactants are separated in a dual or multi chamber bottle and do not recombine until product is dispensed.

Unexpectedly, applicants have found that, if a non-aqueous carrying environment is used, the components generating gas or foam, which are non-reactant with one another in a non-aqueous medium, can be used in a single chamber dispenser. As such, they will not combine until the non-aqueous liquid is diluted with water and foam will thus be generated in use.

In one embodiment, the invention thus comprises a non-aqueous personal wash cleanser comprising:
(1) from 0% to no more than about 25% by wt., preferably no more than about 10%, more preferably no more than about 7% of a surfactant;
(2) from 15 to 50% by wt. (combined percentages of two or more reactants) of a foam enhancing system comprising two or more reactants which are non-reactive with one another (preferably insoluble or sparingly soluble) in the non-aqueous carrying medium of the invention but which, when diluted in water, generate a level of at least 25 cc, more preferably at least 30 cc foam after 30 seconds when measured using defined foam measurement profile of the invention; and
(3) 30 to 70%, preferably 30 to 60% by wt. of a non-aqueous medium defined by the fact that reactants of (2) are non-reactant with each other in said medium.

In a preferred embodiment, polyalkylene glycol (PAG), i.e., 0.1% to 10% by wt. PAG, may be used to accelerate and enhance level of foam produced upon dilution.

Other optional ingredients (including for example thickeners) will be described in detail herein.

In a second embodiment, the invention relates to process for delivering a foam enhancing system to a personal wash cleanser which process or method comprises using reactants forming the foam enhancing system in a non-aqueous medium.

The components of the cleanser compositions are described in more detail below.

Surfactants

One of the advantages of the composition of the invention is that little or even no surfactant may be used. Thus, the irritancy which may be caused by surfactant may be minimized or eliminated altogether.

The surfactant of the invention, if used, may be any surfactant such as are well known to those of ordinary skill in the art and may be selected from the group consisting of anionic, nonionic, amphoteric, zwitterionic and cationic surfactants and mixtures thereof.

Anionic surfactants may include, for example, aliphatic sulfonates such as primary alkane (e.g., C₈-C₂₂) sulfonate or aromatic sulfonates such as alkyl benzene sulfonate. They may further include alkyl sulfates including alkyl ether sulfates.

Anionics may further include alkyl sulfosuccinates, alkyl and acyl taurates, alkyl and acyl sarcosinates, sulfoacetates, alkyl phosphates and their esters, acyl lactates, alkyl succinates and maleates, sulphoacetates and acyl isethionates.

Zwitterionic surfactants are exemplified by those which can be broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. A general formula for these compounds is:

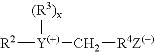

wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Amphoteric detergents which may be used in this invention include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula:

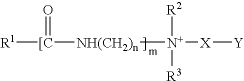

where R¹ is alkyl or alkenyl of 7 to 18 carbon atoms;

R² and R³ are each independently alkyl, hydroxyalkyl or carboxyalkyl of 1 to 3 carbon atoms;

n is 2 to 4;

m is 0 to 1;

X is alkylene of 1 to 3 carbon atoms optionally substituted with hydroxyl, and

Y is —CO₂— or —SO₃—

Suitable amphoteric detergents within the above general formula include simple betaines of formula:

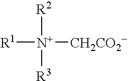

and amido betaines of formula:

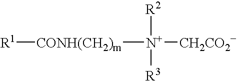

where m is 2 or 3.

In both formulae R¹, R² and R³ are as defined previously. R¹ may in particular be a mixture of C₁₂ and C₁₄ alkyl groups derived from coconut so that at least half, preferably at least three quarters of the groups R¹ have 10 to 14 carbon atoms. R² and R³ are preferably methyl.

The nonionic which may be used includes in particular the reaction products of compounds having a hydrophobic group and a reactive hydrogen atom, for example aliphatic alcohols, acids, amides or alkyl phenols with alkylene oxides, especially ethylene oxide either alone or with propylene oxide. Specific nonionic detergent compounds are alkyl (C₆-C₂₂) phenols-ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxides.

The nonionic may also be a sugar amide, such as a polysaccharide amide. Specifically, the surfactant may be one of the lactobionamides described in U.S. Pat. No. 5,389,279 to Au et al. which is hereby incorporated by reference or it may be one of the sugar amides described in U.S. Pat. No. 5,009,814 to Kelkenberg, hereby incorporated into the subject application by reference.

Other surfactants which may be used are described in U.S. Pat. No. 3,723,325 to Parran Jr. and alkyl polysaccharide nonionic surfactants as disclosed in U.S. Pat. No. 4,565,647 to Llenado, both of which are also incorporated into the subject application by reference.

Preferred alkyl polysaccharides are alkylpolyglycosides of the formula
R²O(CₙH₂ₙO)ₜ(glycosyl)ₓ

wherein R² is selected from the group consisting of alkyl, alkylphenyl, hydroxyalkyl, hydroxyalkylphenyl, and mixtures thereof in which alkyl groups contain from about 10 to about 18, preferably from about 12 to about 14, carbon atoms; n is 0 to 3, preferably 2; t is from 0 to about 10, preferably 0; and x is from 1.3 to about 10, preferably from 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1-position and the preceding glycosyl units 2-, 3-, 4- and/or 6-position, preferably predominantly the 2-position.

Foam Enhancing System

The foam enhancing system of the invention comprises two or more reactants which reactants are non-reactive with one another in the non-aqueous carrying medium of the invention. If the reactants were reactive in the medium, (e.g., if they were in a solubilizing aqueous medium), they would react with one another to gas or foam in the container and prior to the ability of a consumer to be able to take advantage of such foaming upon washing. However, since the reactants are non-reactive, they will not react until the non-aqueous medium is diluted (e.g., upon wash) and the consumer gains the advantage of the foaming.

When the reactants are combined according to the test protocol of the invention, they will foam at a level of at least 25 cc, preferably at least 30 cc after 30 seconds.

One example of a foam enhancing system that can be used in a single compartment medium is the use of alkali metal bicarbonate (e.g., sodium bicarbonate) as one reactant and a C₄-C₂₄ alkyl carboxylic acid (e.g., mono-, di-, or triacids) as the other reactant. One example of a monoacid is lauric acid and one example of a triacid is citric acid. Were such ingredients together in an aqueous medium, they would react immediately to foam or gas. However, when used in the non-aqueous medium of the invention in which they are non-reactant, no reaction occurs until the liquid medium is dispersed and diluted in wash.

Typically, one reactant will comprise 2 to 25% of the final formulation and the other 5 to 30% of the final formulation such that, together they will form 7 to 55% of the final formulation.

It is possible, as noted to use systems also with three reactants and even more as long as they are non-reactive in the non-aqueous medium in which they are found.

Non-Aqueous Carrying Medium

The liquid medium in which the foam reactants are found and are not reactive with one another is a non-aqueous medium.

This can be a predominantly hydrophobic liquid such as an oil or blend of two or more oils or it may be, for example, a liquid glycol. The way is that the reactants should be non-reactive in the medium. While not wishing to be bound by theory, preferably, the medium is one from which the reactants can “come out” upon dilution so they are free to react and foam.

The non-aqueous emulsion generally will comprise about 30 to 70%, preferably 30 to 60% of composition.

In a preferred embodiment of the invention, the invention comprises about 1 to 15%, preferably 2 to 10% by wt. of a polyalkylene glycol such as polyethylene or polypropylene glycol, such as polyethylene or polypropylene glycol. The glycol helps to enhance foam volume expansion.

Optional

One optional ingredient which may be used in the non-aqueous medium are structurants or thickeners.

Particularly, if the medium has some hydrophilic characteristic, suitable thickeners can include electrolytes, various water soluble polymers tolerant to any surfactant which might be present and inorganic materials (e.g., clays or amorphous silica).

If the medium is more hydrophobic, thickeners might include oil soluble polymers, waxes, and inorganic materials (e.g., fumed silica). Materials which are hydrophobically modified may also be used.

Further examples of structurants and thickeners are given in the International Cosmetic Ingredient Dictionary, Fifth Edition, 1993, published by CTFA (The Cosmetic, Toiletry & Fragrance Association), incorporated herein by reference.

Other typical components of such compositions may preferably include opacifiers, preferably 0.05 to 0.5 wt. %, preservatives, preferably 0.05 to 1.0 wt. %, and perfumes, preferably 0.1 to 0.5 wt. %.

Except in the operating and comparative examples, or where otherwise explicitly indicated, all numbers in this description indicating amounts or ratios of materials or conditions or reaction, physical properties of materials and/or use are to be understood as modified by the word “about”.

Where used in the specification, the term “comprising” is intended to include the presence of stated features, integers, steps, components, but not to preclude the presence or addition of one or more features, integers, steps, components or groups thereof.

The following examples are intended to further illustrate the invention and are not intended to limit the invention in any way.

Unless indicated otherwise, all percentages are intended to be percentages by weight. In addition, all ranges are intended to include not only the top and bottom of the range, but all numbers subsumed with the top and bottom of the range

Protocol

Foam Measurements

Foam/gas generation was measured as follows:

1.0 g. of formulation+5.0 g D.I. water were combined in a 100 ml. volumetric flask. Reactants were stirred @ 4 on dial with a magnetic stirrer plate and the volume was measured as noted.

### EXAMPLES

The 4 compositions in Table 1 below were prepared as follows:

The citric acid and lauric acid (if any) were first mixed in the Oil blend or Propylene Glycol. The anhydrous sodium bicarbonate was then added with more mixing. Finally, fumed silica and PEG were added, which were used to ensure that the reactants remain suspended.

TABLE 1
Formulation

| | Conc. % | Conc. % | Conc. % | Conc. % |
|---|---|---|---|---|
| | Formu- | Formu- | Formu- | Formu- |
| Compound Name | lation #1 | lation #2 | lation #3 | lation #4 |
| Sulfosuccinate | 5.0 | 0.0 | 5.0 | 0.0 |
| Citric Acid/ | 15.0 | 7.5 | 15.0 | 7.5 |
| (Anhydrous) | | | | |
| Lauric Acid | 0.0 | 12.5 | 0.0 | 12.5 |
| Sodium Bicarbonate | 20.0 | 20.0 | 20.0 | 20.0 |
| (Anhydrous) | | | | |
| Oil Blend as mentioned | 53.0 | 53.0 | 0.0 | 0.0 |
| in Table 2 below | | | | |
| Propylene Glycol | 0.0 | 0.0 | 53.0 | 53.0 |
| PEG | 5.0 | 5.0 | 5.0 | 5.0 |
| Hydrophobic Fume | | | 2.0 | 2.0 |
| Silica | | | | |
| Hydrophilic Fume | 2.0 | 2.0 | | |
| Silica | | | | |

The oil blend was as noted in Table 2 below:

TABLE 2
Oil Blend

| Oil | Concentration in Percent |
|---|---|
| Soybean Oil | 11.48 |
| Canola Oil | 44.26 |
| Light Mineral Oil | 44.26 |

Testing the 4 compositions for foam generation upon dilution, the following results were observed.

TABLE 3
Foam Volume Expansion
1.0 g. of formulation + 5.0 g D.I. water were combined in a 100 ml.
volumetric flask. Reactants were stirred @ 4 on dial with a magnetic
stirrer plate and the volume was measured as noted.

| Foam Volume (in cubic | Formu- | Formu- | Formu- | Formu- |
|---|---|---|---|---|
| centimeters, or cc) | lation 1 | lation 2 | lation 3 | lation 4 |
| Volume at 30 seconds | 35 cc | 30 cc | 35 cc | 30 cc |
| Volume at 60 seconds | 35 cc | 30 cc | 35 cc | 30 cc |
| (Maximum) | | | | |

As can be seen from the test, all formulations had little or no surfactant (0% or 5% by wt.) and yet all achieved foam of at least 30 after 30 seconds.

1. A non-aqueous personal wash composition comprising:
(a) 0 to 25% by wt. surfactant;
(b) 15 to 50% by wt. of a foam enhancing system comprising two or more reactants non-reactive in a non-aqueous medium in which they are carried, but which will react, when diluted in water, to generate at least 25 cc foam after 30 seconds;
(c) 30 to 70% of said non-aqueous medium.

2. A composition according to claim 1 comprising 0.5 to 25% by wt. surfactant.

3. A composition according to claim 1, wherein foam enhancing system comprises:
(a) alkalimetal bicarbonate; and
(b) C₄-C₂₄ fatty acid.

4. A composition according to claim 1, wherein foam is measured by stirring formulation and water in a container for at least about 30 seconds.
